(19) United States
(12) Patent Application Publication
(10) Pub. No.: US 2024/0352470 A1
(43) Pub. Date: Oct. 24, 2024

(54) COMPOSITION INCLUDING PROBIOTICS FOR TELOMERASE PRODUCTION

(71) Applicant: THE TELOMERASE COMPANY, LLC, LAS VEGAS, NV (US)

(72) Inventor: Jason Ryan Steinbrunn, New Ringgold, PA (US)

(21) Appl. No.: 18/612,750

(22) Filed: Mar. 21, 2024

Related U.S. Application Data
(60) Provisional application No. 63/497,424, filed on Apr. 20, 2023.

(51) Int. Cl. C12N 15/74; A61K 35/747; C12N 1/20; C12N 9/12

(52) U.S. Cl. CPC C12N 15/746; A61K 35/747; C12N 1/205; C12N 9/1276; C12N 2510/00; C12Y 207/07049

(57) ABSTRACT

A composition that comprises recombinant probiotic organisms recombinantly engineered to constitutively express an enzyme involved in maintaining the length of telomeres, wherein the enzyme comprises telomerase subunits, the genetic modification comprises an operon that includes a promoter recognized by the probiotic organism's RNA polymerase and a gene sequence encoding telomerase with genetic modifications that induces secretion of telomerase into a host organism, and the genetic modification of the telomerase gene sequence enhances the telomerase enzyme entrance into the host organism's cells.

### CROSS-REFERENCE TO RELATED APPLICATIONS

This patent application claims priority to provisional patent application No. 63/497,424 filed on Apr. 20, 2023. The subject matter of patent application No. 63/497,424 is incorporated by reference in entirety.

### STATEMENT REGARDING FEDERALLY SPONSORED RESEARCH OR DEVELOPMENT

### Not Applicable.

### INCORPORATION BY REFERENCE OF MATERIAL SUBMITTED ON A COMPACT DISC

Not Applicable.

### TECHNICAL FIELD

The claimed embodiments relate generally to health supplements, and more specifically, to compositions including probiotics for enhancing telomerase production.

### BACKGROUND

The field of longevity and health maintenance has long been a focus of biomedical research, particularly in the context of cellular aging and its impact on overall health. Central to the aging process is the gradual shortening of telomeres, the protective caps at the ends of chromosomes, which occurs with each cell division. Telomere length is a critical factor in cellular senescence, the point at which cells cease to divide. As such, maintaining or extending telomere length is of significant interest for promoting health and potentially extending lifespan.

Historically, the approach to modulating telomere length has predominantly focused on direct genetic interventions, pharmaceuticals, and various lifestyle modifications, each with varying degrees of success and practicality. The enzyme telomerase has been identified as a key player in telomere length maintenance, capable of adding telomeric DNA to the ends of chromosomes and thus counteracting the natural process of telomeric attrition. However, effective and safe methods of enhancing telomerase activity in humans have remained elusive.

Current strategies for telomere length maintenance face several challenges, including the delivery of therapeutic agents in a manner that is both effective and safe, targeting the therapy to the appropriate cells, and achieving sustained telomerase activity without adverse effects. Furthermore, there is a need for interventions that are easily accessible, non-invasive, and compatible with everyday life, which many current approaches do not adequately address.

The prior art reveals attempts at directly manipulating telomerase activity through gene therapy and pharmacological agents, but these methods often come with significant risks, such as increased potential for oncogenesis, and practical limitations in terms of delivery and specificity of action. Additionally, the use of synthetic compounds or heavily engineered biological agents raises concerns about long-term safety and regulatory approval.

Thus, there is a clear unmet need in the prior art for innovative solutions that can safely and effectively modulate telomerase activity, thereby addressing the fundamental challenge of telomere attrition in cellular aging.

### SUMMARY

This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

The disclosed embodiments are directed to a composition comprising recombinant probiotic organisms engineered to constitutively express an enzyme involved in maintaining the length of telomeres, wherein the enzyme consists of telomerase subunits, the genetic modification comprises an operon that includes a promoter recognized by the probiotic organism's RNA polymerase and a gene encoding telomerase, the bacterium includes a genetic modification that induces secretion of telomerase into a host organism and the genetic modification of the telomerase gene sequence enhances the telomerase enzyme entrance into the host organism's cells

To the accomplishment of the above and related objects, claimed subject matter may be embodied in the form illustrated in the accompanying drawings, attention being called to the fact, however, that the drawings are illustrative only, and that changes may be made in the specific construction illustrated and described within the scope of the appended claims. The foregoing and other features and advantages of the claimed embodiments will be apparent from the following more particular description of the preferred embodiments, as illustrated in the accompanying drawings.

### BRIEF DESCRIPTION OF THE DRAWINGS

The accompanying drawings, which are incorporated in and constitute part of this specification, illustrate embodiments of the claimed subject matter and together with the description, serve to explain the principles of the disclosed embodiments. The embodiments illustrated herein are presently preferred, it being understood, however, that the claimed subject matter is not limited to the precise arrangements and instrumentalities shown, wherein:

FIG. 1 is a drawing depicting the composition including probiotics for enhancing telomerase production, in a powder form, according to one embodiment;

FIG. 2 is a drawing depicting the composition including probiotics for enhancing telomerase production, in a capsule form, according to one embodiment;

FIG. 3 is a drawing depicting the composition including probiotics for enhancing telomerase production, in a capsule form, according to one embodiment;

FIG. 4 is a drawing depicting the composition including probiotics for enhancing telomerase production, in various ready-to-eat food forms, according to one embodiment.

### DETAILED DESCRIPTION

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions or modifications may be made to the elements illustrated in the drawings, and the methods described herein may be modified by substituting, reordering, or adding additional stages or components to the disclosed methods and devices. Accordingly, the following detailed description does not limit the disclosed embodiments. Instead, the proper scope of the disclosed embodiments is defined by the appended claims.

The claimed embodiments described herein enhance the body's capability to fight aging, among other things. The claimed embodiments mark a significant advancement over prior art in the field of longevity and health maintenance, particularly addressing the challenge of telomere attrition, a critical factor in cellular aging. Traditional approaches to maintaining or extending telomere length have predominantly focused on direct genetic interventions, pharmaceuticals, and various lifestyle modifications. These methods, while offering some benefits, have been limited by issues related to delivery, targeting, safety, and practicality. Genetic therapies and pharmacological agents, for instance, have faced hurdles due to their potential for oncogenesis, difficulty in targeting specific cells, and the challenges of achieving sustained activity without adverse effects. Moreover, the reliance on synthetic compounds or heavily engineered biological agents has raised concerns about long-term safety and regulatory approval.

The claimed embodiments provide an innovative solution by leveraging recombinant probiotic organisms engineered to constitutively express telomerase, the enzyme responsible for adding DNA sequences to the ends of chromosomes, thus counteracting telomere shortening. Unlike previous methods, this approach offers a non-invasive, easily accessible, and potentially safer alternative for enhancing telomerase activity in humans. By incorporating the genetic modification into probiotic organisms that are already recognized for their health benefits, the claimed embodiments solve the problem of delivery and specificity by utilizing the natural mechanism of probiotics to interact with and influence host biological systems. This ensures that the therapeutic agent, telomerase, is produced and secreted directly into the host organism, targeting cells more effectively and reducing the risk of adverse effects associated with other delivery methods.

Furthermore, the use of probiotics circumvents the limitations associated with synthetic compounds and gene therapy, offering a solution that is more likely to be accepted by regulatory bodies and perceived as safe by the public. The versatility of the formulation, including options for incorporation into foods, beverages, or direct supplementation, addresses the practicality and accessibility issues of previous approaches, making it easier for individuals to integrate this solution into their daily routines.

By addressing the fundamental challenge of telomere attrition through a novel, probiotic-based strategy, the claimed embodiments not only overcome the significant limitations of the prior art but also open new approaches for promoting health and longevity. The claimed embodiments' approach to enhancing telomerase activity in a safe, effective, and user-friendly manner represent significant advancements in the field of biomedicine and longevity research.

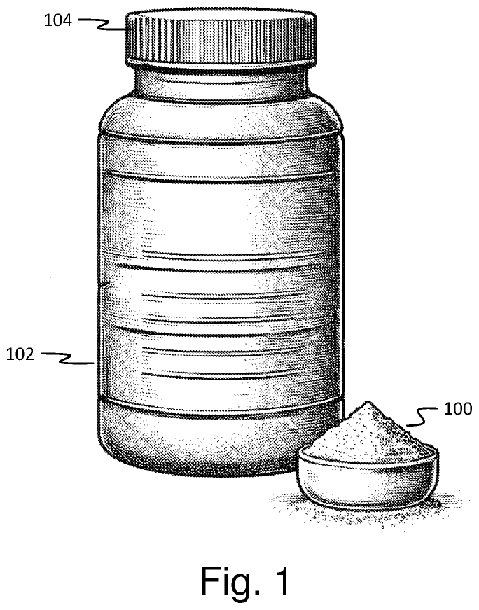

FIG. 1 is a drawing depicting the composition 100 including probiotics for enhancing telomerase production, in a powder form, according to one embodiment. FIG. 1 shows that the composition may be provided in powder form 100 to consumers within a container 102 having a removable top 104 for gaining access to the interior of the container. Said powder may be ingested by mixing with a liquid such as water or milk or may be added to solid food.

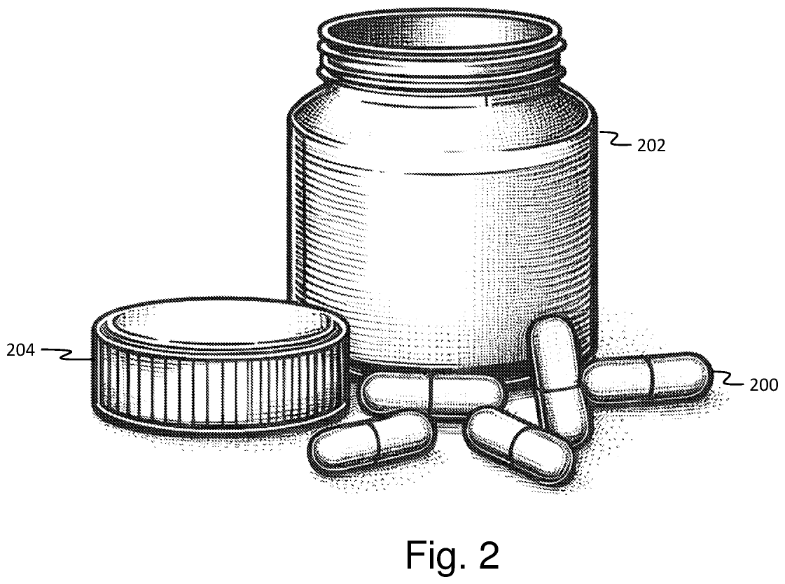

FIG. 2 is a drawing depicting the formulation for the composition 100 including probiotics for enhancing telomerase production, in a capsule form, according to one embodiment. FIG. 2 shows that the composition may be provided in a capsule form 200 to consumers within container 202 having a removable top 204 for gaining access to the interior of the container.

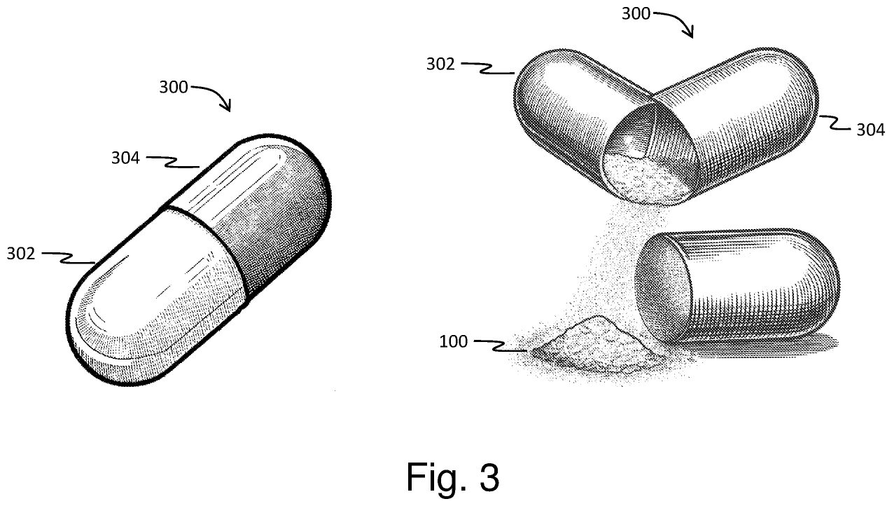

FIG. 3 is a drawing depicting the composition 100 including probiotics for enhancing telomerase production, in a capsule form, according to one embodiment. FIG. 3 shows that the composition 100 is provided in a capsule 300 that includes a first portion 302 and a second portion 304, wherein the composition 100 is present in a powder form within the capsule. The design of the capsule 300, including its composition, shell thickness, and any special coatings, can affect the release rate and absorption characteristics of the composition 100. Some capsules are designed for immediate release, while others are formulated for sustained or delayed release to provide a longer-lasting effect.

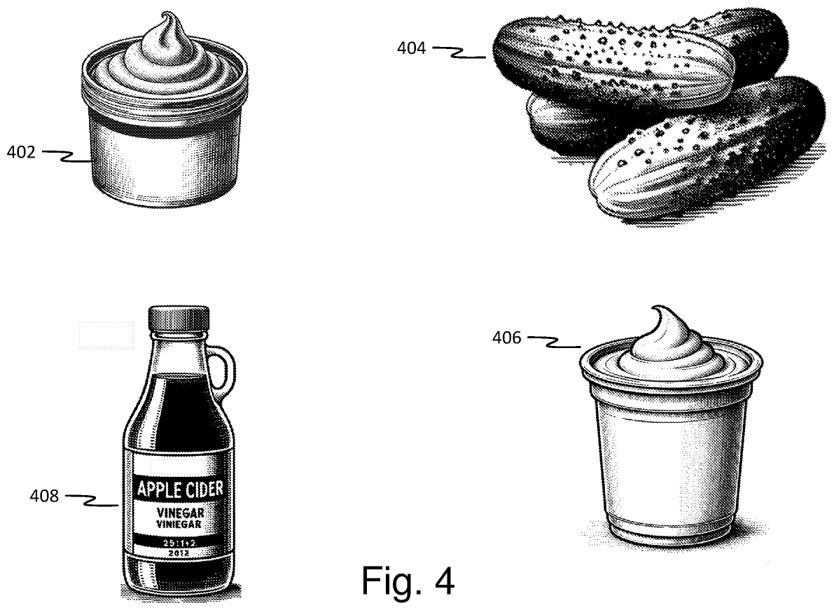

FIG. 4 is a drawing depicting the composition 100 including probiotics for enhancing telomerase production, in various ready-to-eat food forms, according to one embodiment. FIG. 4 shows that the composition may be provided in various ready-to-eat food forms, wherein the composition 100 is present in, for example, sour cream 402, fermented pickles 404, apple cider vinegar 408 and yogurt 406. Additional examples of ready-to-eat food forms include brine cured olives, cheese, cottage cheese, cultured buttermilk, kefir, kimchi, kombucha, koumiss, kvas, miso, natto, sauerkraut, tempeh, viili yogurt, and water kefir.

The claimed composition 100 stands as a groundbreaking approach to promoting longevity and cellular health through the maintenance of telomere length. The claimed composition comprises genetically engineered probiotic organisms. These organisms have been meticulously designed to constitutively express telomerase, an enzyme pivotal in the elongation and maintenance of telomeres. Telomeres, the protective caps at the end of chromosomes, play a critical role in cellular aging by safeguarding the chromosome ends from deterioration. As cells divide, telomeres gradually shorten, leading to cellular senescence or the loss of a cell's power to divide and grow. This process is a natural part of aging and a key interest area for extending health-span and lifespan.

The genetically engineered probiotic organisms in the claimed composition are created through recombinant DNA technology, which allows for the introduction of new genetic material. The introduced genetic modifications include an operon, a cluster of genes that operate under the control of a single promoter. A promoter is a DNA sequence recognized by the organism's RNA polymerase, initiating the transcription of the gene it controls—in this case, the gene encoding telomerase. This strategic modification ensures the probiotics continually produce telomerase. Furthermore, the bacteria are engineered to secrete telomerase, facilitating its delivery into the host organism and promoting the maintenance of telomere length from within. Note the genetic modification of the telomerase gene sequence enhances the telomerase enzyme entrance into the host organism's cells.

RNA polymerase is a critical enzyme found in all living organisms that plays a fundamental role in the process of transcription, which is the first step in gene expression. During transcription, RNA polymerase reads the DNA template strand and synthesizes a complementary strand of RNA. This RNA strand can be messenger RNA (mRNA), transfer RNA (tRNA), or ribosomal RNA (rRNA), depending on the gene being transcribed. Each type of RNA has a specific function in the cell, with mRNA serving as the template for protein synthesis during translation, tRNA bringing amino acids to the ribosome for protein assembly, and rRNA forming the core of ribosome's structure and catalyzing protein synthesis.

The claimed composition specifies organisms belonging to various genera, a term in biological classification denoting a rank that groups together species exhibiting common characteristics. Selected genera include Bacillus, Bifidobacterium, Enterococcus, Escherichia, and several others within the lactobacillus group, each known for their health-promoting properties. The claimed composition further narrows down to specific species within these genera, such as Bacillus subtilis and Bifidobacterium bifidum, chosen for their proven safety and efficacy as probiotics.

The probiotic organisms in the claimed composition belong to a genus selected from Bacillus, Bifidobacterium, Enterococcus, Escherichia, Fructilactobacillus formerly Lactobacillus, Lacticaseibacillus formerly Lactobacillus, Lactiplantibacillus formerly Lactobacilus, Lactobacillus, Lentilactobacillus formerly Lactobacillus, Levilactobacillus formerly Lactobacillus, Limosilactobacillus formerly Lactobacillus, Loigolactobacillus formerly Lactobacillus, Lactococcus formerly Lactobacillus xylosus, Leuconostoc, Pediococcus, Saccharomyces, Streptococcus, Weissella formerly Lactobacillus, and Yarrowia.

The claimed composition includes probiotic organisms selected from the species Bacillus subtilis, Bacillus amyloliquefaciens, Bifidobacterium animalis, Bifidobacterium bifidum, Bifidobacterium breve, Bifidobacterium infantis, Bifidobacterium longum, Enterococcus faecalis, Enterococcus faecium, Escherichia coli, Fructilactobacillus fructivorans formerly Lactobacillus fructivorans, Lacticaseibacillus casei formerly Lactobacillus casei, Lacticaseibacillus paracasei formerly Lactobacillus paracasei, Lacticaseibacillus rhamnosus formerly Lactobacillus rhamnosus, Lactiplantibacillus pentosus formerly Lactobacillus pentosus. Lactiplantibacillus plantarum formerly Lactobacillus plantarum. Lactobacillus acidophilus, Lactobacillus delbrueckii formerly Lactobacillus Lactis, Lentilactobacillus hilgardii formerly Lactobacillus hilgardii, Lentilactobacillus kefiri formerly Lactobacillus kefiri, Levilactobacillus brevis formerly Lactobacillus brevis, Limosilactobacillus fermentum formerly Lactobacillus fermentum, Limosilactobacillus reuteri formerly Lactobacillus reuteri, Loigolactobacillus coryniformis formerly Lactobacillus coryniformis, Lactococcus lactis formerly Lactobacillus xylosus, Leuconostoc cremoris, Leuconostoc mesenteroides, Leuconostoc pseudomesenteroides, Pediococcus acidilactici, Pediococcus damnosus, Pediococcus pentosaceus, Saccharomyces boulardii, Streptococcus lactis, Streptococcus thermophilus, Weissella cibaria formerly Lactobacillus coprophilus, and Yarrowia lipolytica.

To enhance the expression and functionality of the telomerase enzyme by the claimed composition, additional genetic modifications are introduced. These modifications include nucleic acid sequences encoding transcription factors that regulate the expression of the telomerase gene, ensuring the enzyme is produced efficiently and effectively within the probiotic strain.

The probiotic composition may be formulated into a pharmaceutical composition, incorporating a pharmaceutically acceptable carrier to ensure safety and efficacy. This formulation is designed for versatile administration routes, including topical, oral, or anal, providing flexibility to meet various treatment needs and preferences. A stabilizing agent is also included to enhance the viability of the probiotic organisms during storage and upon administration, ensuring the live bacteria remain active and effective in delivering the telomerase enzyme to the host. The composition may also include a subunit that further enhances enzyme expression, encapsulated in capsule form (as a powder) or formulated into the cultured fermentation of food or beverage (such as sour cream, fermented pickles, apple cider vinegar and yogurt), offering convenience and ease of integration into daily routines. A protein subunit is a polypeptide chain or single protein molecule that assembles or “coassembles” with others to form a protein complex.

For oral administration, the key challenge is to protect the probiotic organisms as they navigate the acidic gastric environment to reach the intestines, where they can effectively colonize and express telomerase. This is typically addressed by employing advanced encapsulation techniques, such as microencapsulation or the use of enteric coatings that dissolve only at the higher pH levels found in the intestines. The formulation may be complemented with prebiotics to nourish and enhance the probiotics' survival and activity, thus ensuring their maximum therapeutic potential.

In the case of topical administration, the formulation strategy focuses on enabling the probiotics and the telomerase enzyme to penetrate the skin barrier. This often involves the use of delivery systems like liposomes or hydrogels that can facilitate absorption through the skin layers. Such formulations aim not only to maintain the viability of the probiotics on the skin's surface but also to ensure the stability and bioactivity of the secreted telomerase, potentially aiding in skin health and anti-aging efforts.

For anal administration, the composition is usually prepared in forms such as suppositories or enemas that allow for the direct delivery of probiotics to the lower gastrointestinal tract. The design of these formulations ensures that they release their contents at body temperature once inserted, placing the probiotics in immediate proximity to their target site. This route can be particularly effective for achieving high localized concentrations of probiotics, enhancing their colonization and therapeutic activity. Across all these administration methods, the formulation is carefully designed to include stabilizers, preservatives, and nutrients that support the probiotics' viability and functionality. Buffering agents may also be incorporated to maintain an optimal pH for the probiotics' survival and activity.

Stabilizing agents used in the claimed composition ensure the biological components retain their viability, activity, and structural integrity from the point of manufacture until administration. Polyols and sugars, such as trehalose, sorbitol, and mannitol, serve as osmoprotectants. They shield proteins and cell membranes from dehydration and protect against osmotic stress during freeze-drying and storage. Proteinaceous substances like skim milk, gelatin, and soy protein act as protectants for probiotic cells and enzymes, forming a protective barrier that mitigates damage through freezing, drying, and rehydration processes. Glycerol, utilized as a cryoprotectant in liquid formulations, guards cells and enzymes against the harms of freezing and thawing by minimizing ice crystal formation and stabilizing cell membranes. Microencapsulation materials, including polymers like alginate, chitosan, and poly-lactic-co-glycolic acid (PLGA), offer a physical barrier around probiotic cells or enzymes. This barrier not only protects against environmental stresses, such as pH and enzymatic degradation but also enables controlled release at the target site. Antioxidants, such as ascorbic acid (vitamin C) and tocopherols (vitamin E), are added to formulations to prevent oxidative damage to probiotic cells and enzymes, which can lead to degradation of cellular components and loss of activity. Buffering agents like phosphate, citrate, or acetate buffers maintain an optimal pH range, crucial for the stability and activity of probiotics and enzymes since extreme PH levels can denature proteins and disrupt cell membranes. Lastly, lyoprotectants including lactose and sucrose are employed in freeze-drying processes. They safeguard the structure and function of the probiotic cells and enzymes during lyophilization and subsequent rehydration.

In the approach of enhancing telomerase expression through recombinant probiotic organisms, the subunit that enhances enzyme expression plays a crucial role. This component is a finely tuned genetic or molecular addition designed to boost the production levels of the telomerase enzyme within the host organism. The enhancement of enzyme expression through such a subunit involves genetic engineering strategies aimed at increasing the activity of telomerase, a key player in extending the length of telomeres and potentially improving cellular longevity. The concept includes incorporating specific promoter sequences into the DNA of probiotics. These sequences serve as crucial starting points for the transcription process, acting as docking sites for RNA polymerase and other transcription factors, thereby determining the rate of gene transcription into messenger RNA (mRNA). The use of strong, constitutively active promoters can lead to a significant uptick in the expression levels of telomerase, ensuring efficient transcription.

Alongside promoters, enhancer sequences play a pivotal role. These DNA elements can be placed at varying distances from the gene they regulate, binding specific proteins that interact with the transcription machinery. The interaction amplifies the transcription rate of the telomerase gene, thus elevating enzyme production. Regulatory elements are also key, offering precision in how telomerase expression is controlled within the host. These sequences allow for the enzyme's production to be adjusted based on environmental cues or specific internal conditions, ensuring that telomerase is produced in a timely and spatially relevant manner. Additionally, codon optimization is a strategy that tailors the gene sequence to align with the codon usage preferences of the host organism, improving the efficiency of the translation process. This adjustment ensures that the messenger RNA is more effectively translated into the telomerase enzyme, enhancing the overall production rate.

In essence, the claimed composition leverages the symbiotic relationship between humans and probiotics to offer a novel, non-invasive solution to the challenge of telomere attrition, a fundamental aspect of cellular aging. By providing a detailed explanation of the scientific principles and methodologies employed, this description underscores the invention's innovative approach to enhancing health and longevity through the maintenance of telomere length.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

1. A composition comprising recombinant probiotic organisms recombinantly engineered to constitutively express an enzyme involved in maintaining the length of telomeres, wherein:
a) the enzyme comprises telomerase subunits;
b) the genetic modification comprises an operon that includes a promoter recognized by the probiotic organism's RNA polymerase and a gene sequence encoding telomerase;
c) the bacterium includes a genetic modification that induces secretion of telomerase into a host organism; and
d) the genetic modification of the telomerase gene sequence enhances the telomerase enzyme entrance into the host organism's cells.

2. The composition of claim 1, wherein said genetic modifications comprise one or more nucleic acid sequences encoding transcription factors that regulate expression of said telomerase genes in said probiotic organism.

3. The composition of claim 2, wherein the probiotic organisms belong to a genus selected from Bacillus, Bifidobacterium, Enterococcus, Escherichia, Fructilactobacillus formerly Lactobacillus, Lacticascibacillus formerly Lactobacillus, Lactiplantibacillus formerly Lactobacilus, Lactobacillus, Lentilactobacillus formerly Lactobacillus, Levilactobacillus formerly Lactobacillus, Limosilactobacillus formerly Lactobacillus, Loigolactobacillus formerly Lactobacillus, Lactococcus formerly Lactobacillus xylosus, Leuconostoc, Pediococcus, Saccharomyces, Streptococcus, Weissella formerly Lactobacillus, and Yarrowia.

4. The composition of claim 2, wherein the probiotic organisms are selected from the species Bacillus subtilis, Bacillus amyloliquefaciens, Bifidobacterium animalis, Bifidobacterium bifidum, Bifidobacterium breve, Bifidobacterium infantis, Bifidobacterium longum, Enterococcus faecalis, Enterococcus faecium, Escherichia coli, Fructilactobacillus fructivorans formerly Lactobacillus fructivorans, Lacticaseibacillus casei formerly Lactobacillus casei, Lacticascibacillus paracasei formerly Lactobacillus paracasei, Lacticaseibacillus rhamnosus formerly Lactobacillus rhamnosus, Lactiplantibacillus pentosus formerly Lactobacillus pentosus, Lactiplantibacillus plantarum formerly Lactobacillus plantarum, Lactobacillus acidophilus, Lactobacillus delbrueckii formerly Lactobacillus Lactis, Lentilactobacillus hilgardii formerly Lactobacillus hilgardii, Lentilactobacillus kefiri formerly Lactobacillus kefiri, Levilactobacillus brevis formerly Lactobacillus brevis, Limosilactobacillus fermentum formerly Lactobacillus fermentum, Limosilactobacillus reuteri formerly Lactobacillus reuteri, Loigolactobacillus coryniformis formerly Lactobacillus coryniformis, Lactococcus lactis formerly Lactobacillus xylosus, Leuconostoc cremoris, Leuconostoc mesenteroides, Leuconostoc pseudomesenteroides, Pediococcus acidilactici, Pediococcus damnosus, Pediococcus pentosaceus, Saccharomyces boulardii, Streptococcus lactis, Streptococcus thermophilus, Weissella cibaria formerly Lactobacillus coprophilus, and Yarrowia lipolytica.

5. The composition of claim 4, wherein the probiotic organisms are formulated in a pharmaceutical composition that further comprises a pharmaceutically acceptable carrier.

6. The composition of claim 5, wherein the pharmaceutical composition is formulated for topical, oral or anal administration.

7. The composition of claim 6, further comprising a stabilizing agent to enhance the viability of the probiotic organisms during storage and upon administration to the host organism.

8. The composition of claim 7, wherein the enzyme telomerase includes a subunit that enhances enzyme expression.

9. The composition of claim 8, wherein the pharmaceutical composition is encapsulated in a capsule form.

10. The composition of claim 9, wherein the pharmaceutical composition is formulated into the cultured fermentation of a food or beverage.

11. A composition comprising recombinant probiotic organisms recombinantly engineered to constitutively express an enzyme involved in maintaining the length of telomeres, wherein:
a) the enzyme comprises telomerase subunits;
b) the genetic modification comprises an operon that includes a promoter recognized by the probiotic organism's RNA polymerase and a gene sequence encoding telomerase;
c) the bacterium includes a genetic modification that optimizes the expression of the telomerase genes; and
d) the genetic modification of the telomerase gene sequence enhances the telomerase enzyme entrance into the host organism's cells.

12. The composition of claim 11, wherein said genetic modifications comprise one or more nucleic acid sequences encoding transcription factors that regulate expression of said telomerase genes in said probiotic strain.

13. The composition of claim 12, wherein the probiotic organisms belong to a genus selected from Bacillus, Bifidobacterium, Enterococcus, Escherichia, Fructilactobacillus formerly Lactobacillus, Lacticaseibacillus formerly Lactobacillus, Lactiplantibacillus formerly Lactobacilus, Lactobacillus, Lentilactobacillus formerly Lactobacillus, Levilactobacillus formerly Lactobacillus, Limosilactobacillus formerly Lactobacillus, Loigolactobacillus formerly Lactobacillus, Lactococcus formerly Lactobacillus xylosus, Leuconostoc, Pediococcus, Saccharomyces, Streptococcus, Weissella formerly Lactobacillus, and Yarrowia.

14. The composition of claim 13, wherein the probiotic organisms are selected from the species Bacillus subtilis, Bacillus amyloliquefaciens, Bifidobacterium animalis, Bifidobacterium bifidum, Bifidobacterium breve, Bifidobacterium infantis, Bifidobacterium longum, Enterococcus faecalis, Enterococcus faecium, Escherichia coli, Fructilactobacillus fructivorans formerly Lactobacillus fructivorans, Lacticaseibacillus casei formerly Lactobacillus casei, Lacticaseibacillus paracasei formerly Lactobacillus paracasei, Lacticascibacillus rhamnosus formerly Lactobacillus rhamnosus, Lactiplantibacillus pentosus formerly Lactobacillus pentosus, Lactiplantibacillus plantarum formerly Lactobacillus plantarum, Lactobacillus acidophilus, Lactobacillus delbrueckii formerly Lactobacillus Lactis, Lentilactobacillus hilgardii formerly Lactobacillus hilgardii, Lentilactobacillus kefiri formerly Lactobacillus kefiri, Levilactobacillus brevis formerly Lactobacillus brevis, Limosilactobacillus fermentum formerly Lactobacillus fermentum, Limosilactobacillus reuteri formerly Lactobacillus reuteri, Loigolactobacillus coryniformis formerly Lactobacillus coryniformis, Lactococcus lactis formerly Lactobacillus xylosus, Leuconostoc cremoris, Leuconostoc mesenteroides, Leuconostoc pseudomesenteroides, Pediococcus acidilactici, Pediococcus damnosus, Pediococcus pentosaceus, Saccharomyces boulardii, Streptococcus lactis, Streptococcus thermophilus, Weissella cibaria formerly Lactobacillus coprophilus, and Yarrowia lipolytica.

15. The composition of claim 14, wherein the probiotic organisms are formulated in a pharmaceutical composition that further comprises a pharmaceutically acceptable carrier.

16. The composition of claim 15, wherein the pharmaceutical composition is formulated for topical, oral or anal administration.

17. The composition of claim 16, further comprising a stabilizing agent to enhance the viability of the probiotic organisms during storage and upon administration to the host organism.

18. The composition of claim 17, wherein the enzyme telomerase includes a subunit that enhances enzyme expression.

19. The composition of claim 18, wherein the pharmaceutical composition is encapsulated in a capsule form.

20. The composition of claim 19, wherein the pharmaceutical composition is formulated into the cultured fermentation of a food or beverage.
